(19) United States
(12) Patent Application Publication
(10) Pub. No.: US 2026/0260341 A1
(43) Pub. Date: Sep. 3, 2026

(54) FORECASTING SHAPES OF TISSUE REGIONS USING MACHINE LEARNING

(71) Applicant: UNIVERSITY OF PITTSBURGH - OF THE COMMONWEALTH SYSTEM OF HIGHER EDUCATION, Pittsburgh, PA (US)

(72) Inventors: Timothy K. Chung, Pittsburgh, PA (US); David A. Vorp, Pittsburgh, PA (US); Aakash Kottakota, Pittsburgh, PA (US); Jason Lee, Pittsburgh, PA (US); Pete Gueldner, Pittsburgh, PA (US)

(21) Appl. No.: 19/163,497

(22) PCT Filed: Mar. 8, 2024
(86) PCT No.: PCT/US2024/019084

§ 371 Date: Sep. 9, 2025

Related U.S. Application Data
(60) Provisional application No. 63/451,097, filed on Mar. 9, 2023.

(51) Int. Cl. G06T 7/00; G06T 7/11; G16H 30/40; G16H 50/50

(52) U.S. Cl. CPC G06T 7/0012; G06T 7/11; G16H 30/40; G16H 50/50; G06T 2207/30024

(57) ABSTRACT

Methods, systems, and apparatus, including computer programs encoded on a computer storage medium, for generating a predicted surface model of a tissue region. In one aspect, a method comprises: obtaining a surface model that defines a surface of a tissue region in a subject at a first time point; generating a predicted surface model that defines a predicted surface of the tissue region in the subject at a second time point using a machine learning model, comprising: generating a model input to the machine learning model based on the surface model of the tissue region for first time point; and processing the model input using the machine learning model, in accordance with values of a set of machine learning model parameters, to generate a model output that specifies the predicted surface model of the tissue region in the subject at the second time point.

### CROSS-REFERENCE TO RELATED APPLICATIONS

This application claims the benefit of priority to U.S. Provisional Application Ser. No. 63/451,097, filed Mar. 9, 2023, the contents of which are incorporated by reference in their entirety.

### STATEMENT REGARDING FEDERALLY SPONSORED RESEARCH

This invention was made with government support under HL156246 awarded by the National Institutes of Health. The government has certain rights in the invention.

### BACKGROUND

### 1. Technical Field

This specification describes machine-learning techniques for predicting the surface shapes of tissue regions (e.g., aneurysms, diseased vasculature, tumors, or cysts) at future time points.

### 2. Background

Machine learning models receive an input and generate an output, e.g., a predicted output, based on the received input. Some machine learning models are parametric models and generate the output based on the received input and on values of a set of parameters of the model (e.g., weights of nodes in an artificial neural network that are automatically learned through a training procedure).

Some machine learning models are deep models that employ multiple layers of models to generate an output for a received input. For example, a deep neural network is a deep machine learning model that includes an output layer and one or more hidden layers that each apply a non-linear transformation to a received input to generate an output. Another example is a recurrent neural network (e.g. long short-term memory). Machine-learning models can be used in artificial intelligence (AI) systems that learn from experience to perform tasks without the need for explicit programming from a human.

Abdominal aortic aneurysm (AAA) is a leading cause of death in westernized countries and affects an aging population. The adult abdominal aorta is typically 2 centimeters (cm) in diameter, and is defined as aneurysmal when the diameter grows to exceed 3 cm. If left untreated, AAA may continue to grow and eventually rupture, with resulting morbidity and mortality rates exceeding 85%. Cerebral aneurysm is also a leading cause of death globally, and affect a wide range of patient demographics based on age, sex, and locale within the cerebrovasculature.

### SUMMARY

This specification describes a shape forecasting system that can be implemented as computer programs on one or more computers in one or more locations. The shape forecasting system can process features of a surface model that defines a surface of a tissue region in a subject at a first time point using a machine learning model to generate a predicted surface model of the tissue region at a second time point.

Throughout this specification, a “point,” e.g., on the surface of a tissue region shown in a medical image, can be represented in any appropriate coordinate system, e.g., a Cartesian coordinate system, a polar coordinate system, or a spherical coordinate system.

Particular embodiments of the subject matter described in this specification can be implemented so as to realize one or more of the following advantages.

The shape forecasting system described in this specification can accurately forecast changes in the surface shape of tissue regions such as aneurysms, diseased vasculature, tumors, and cysts. The shape forecasting system can thus improve patient outcomes by enabling the progression of diseases and pathologies to be accurately predicted and promptly addressed through appropriate treatment.

The shape forecasting system can predict future (or past) surface shapes of a tissue region using a machine learning model that can be trained to perform implicit spatial and geometric reasoning across a large number (e.g., thousands) of densely sampled points distributed across the surface of the tissue region. The shape forecasting system can thus achieve greater accuracy than conventional techniques for forecasting progression in the shapes of tissue regions, e.g., that rely on criteria that are manually specified by human experts and are often basic in nature. In contrast, the shape forecasting system provides a data-driven approach for automatically forecasting surface shapes of tissue regions based on complex patterns and correlations in geometric data well beyond what could be analyzed by a human or solely in the human mind.

The details of one or more embodiments of the subject matter of this specification are set forth in the accompanying drawings and the description below. Other features, aspects, and advantages of the subject matter will become apparent from the description, the drawings, and the claims.

### BRIEF DESCRIPTION OF THE DRAWINGS

FIG. 1 shows an example environment that includes a medical imaging system, a modeling system, and a shape forecasting system.

FIG. 2 shows an example shape forecasting system.

FIG. 3 is a flow diagram of an example process for predicting a surface model for a tissue region at a second time point based on a surface model for the tissue region at a first time point.

FIG. 4 is a flow diagram of an example process for training a machine learning model that processes a model input based a surface model of a tissue region at a first time point to generate a model output that specifies a predicted surface model of the tissue region at a second time point.

FIG. 5 depicts a progression of surface models of an abdominal aortic aneurysm (AAA) over time. Known data was utilized to generate models depicting growth of the aneurysm from 6 to 36 months with 6-month intervals in between. The heat map indicates localized displacements of points that show various rates of growth across the aneurysm for an intuitive display.

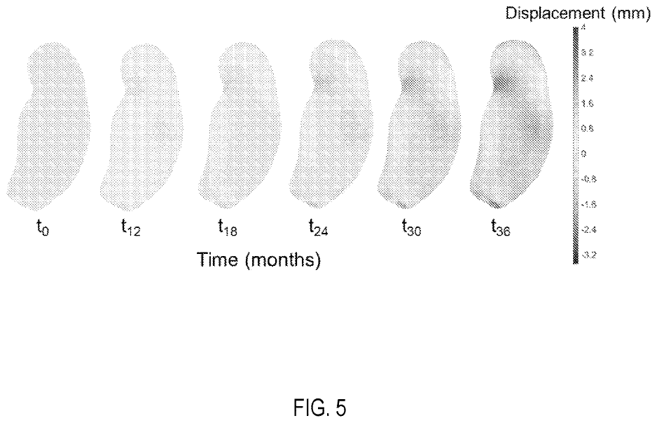

FIG. 6A depicts a comparison of a predicted surface model and an actual surface model (e.g., a target output) of an AAA in a study of an actual machine-learning model implemented according to the techniques disclosed in this specification. FIG. 6B is a table of values that indicate the accuracy of the predicted surface model with respect to the actual surface model (ground truth).

FIG. 7 depicts the transformation of an example AAA surface model over time. An initial (baseline) surface model of the patient's AAA is shown at the left. Evolved surface models of the AAA at respective time points 25 and 48 weeks after the AAA was initially modeled are shown at the right. The top right portion of the image specifically shows the ground truth surface models of the AAA at 25 and 48 weeks, respectively. These models were created based on new medical images obtained at 25 and 48 weeks after the initial scan. The bottom right portion of the image shows surface models of the AAA at 25 and 48 weeks as predicted by a neural network machine-learning model implemented in accordance with the techniques described herein. For example, the machine-learning model can process the baseline AAA surface model to evolution of the AAA at 25 weeks, and can then process the predicted 25-week model to generate the predicted 48-week model.

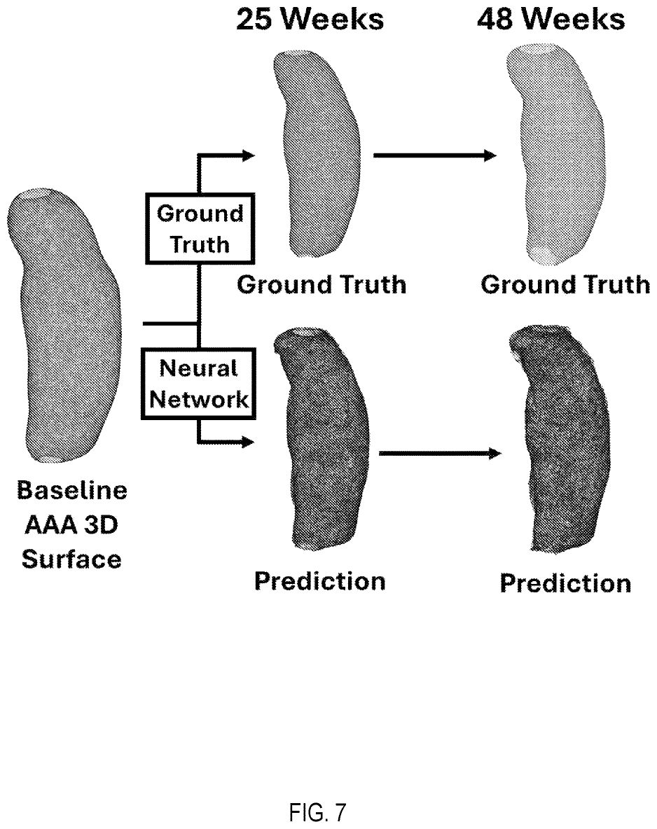

Like reference numbers and designations in the various drawings indicate like elements.

### DETAILED DESCRIPTION

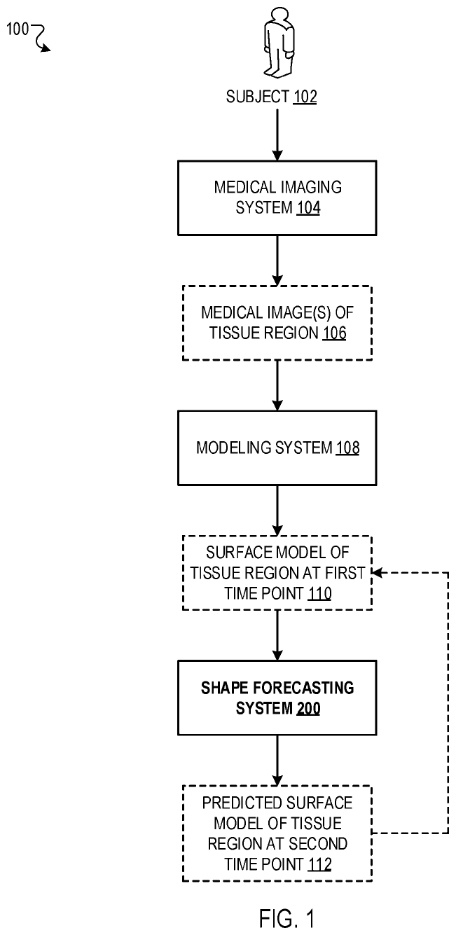

FIG. 1 shows an example environment 100 that includes a medical imaging system 104, a modeling system 108, and a shape forecasting system 200, which are each described in more detail next.

The medical imaging system 104 can generate one or more medical images 106 of a subject 102 using an appropriate medical imaging modality. For example, the medical imaging system 104 may produce a set of medical images 106 in which each image depicts a slice of the subject's anatomy at a different location along a defined dimension or axis of the subject 102.

The subject 102 can be a person or an animal, e.g., a mouse, a cat, a dog, a rabbit, a pig, or other mammalian or non-mammalian subject.

The medical imaging modality can be, e.g., a magnetic resonance imaging (MRI) modality, a computed tomography (CT) modality, an ultrasound (US) modality, an x-ray modality, etc.

The medical images 106 can show a region of tissue in the subject, for instance, a tumor, an aneurysm, diseased vasculature, or a cyst. A tumor can refer to a mass of tissue resulting from abnormal and excessive tissue growth. An aneurysm can refer to an outward bulging of a blood vessel, e.g., caused by a localized, abnormal, weak spot on the wall of the blood vessel. A cyst can refer to a hollow organ or cavity containing a liquid secretion. A diseased vasculature can include a stenotic artery or damaged vein with low compliance (e.g. deep vein thrombosis). The tissue region can be located in any appropriate part of the subject, e.g., the brain of the subject, the thorax of the subject, the abdomen of the subject, etc. The medical images 106 can include, e.g., a collection of one or more two-dimensional (2D) medical image, one or more three-dimensional (3D) medical image, or both. A given medical image 106 can be represented, e.g., as an ordered collection of pixels, where each pixel has an associated intensity value.

The modeling system 108 can process one or more medical image(s) 106 of the subject to generate data defining the surface of the tissue region in the subject (a “surface model”). The “surface” of the tissue region can refer to a boundary of the tissue region, e.g., the outermost boundary of the tissue region. The surface of the tissue region can, for example, outline the boundary of the tissue region in 2D space (e.g., if the medical image is a 2D image) or in 3D space (e.g., if the medical image is a 3D image).

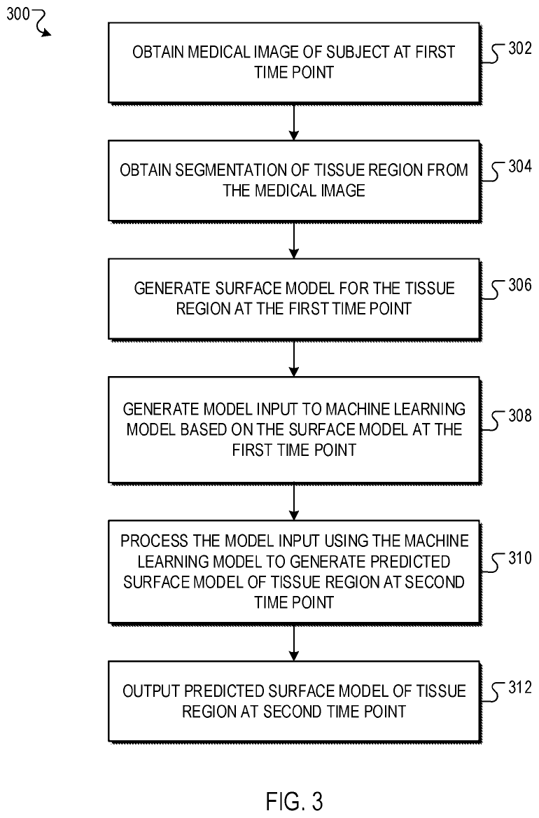

The surface model of the tissue region can be represented in any appropriate manner. For instance, the surface model can be represented as a point cloud, e.g., a collection of points (“surface points”) located on the surface of the tissue region. As another example, the surface model can be represented as a polygon mesh, e.g., a collection of vertices, edges, and faces that cover the surface of the tissue region. The faces of the polygon mesh can be, e.g., triangles (such that the mesh is a triangular mesh), quadrilaterals (such that the mesh is a quadrilateral mesh), or any other appropriate shape. The surface model can be represented as a non-uniform rational B-splines (NURBS), a mathematical expression of the 3D geometry. The surface of the tissue region can be obtained, e.g., by segmenting the tissue region from the medical image 106 of the subject 102, as will be described in more detail with reference to FIG. 3.

Generally, the surface model of the tissue region 110 characterizes the tissue region at a particular point in time, which for convenience can be referred to as a “first time point.” The first time point can be, e.g., the time point at which the medical imaging system 104 captured the medical image of the tissue region in the subject.

The shape forecasting system 200 is configured to process the surface model of the tissue region at the first time point 110 to generate data defining a predicted surface of the tissue region at a second time point 112. The predicted surface of the tissue region can be represented in any appropriate form, including in a same or different form as the input that represents the first model of the tissue region 110 at the first time point (e.g., the predicted surface can be represented as a collection of surface points located on the surface of the tissue region at the second time point 112 or as a polygon mesh that define portions of the predicted surface of the tissue region at the second time point 112). The shape of the tissue region in the subject 102 may change over time, e.g., for underlying physiological or biomechanical reasons. For instance, the shape of a tumor may change over time, e.g., as the tumor grows as a result of excess cell division or shrinks as a result of medical treatment. As another example, the shape of an aneurysm may change over time, e.g., as the force of flowing blood distends the aneurysm.

The shape forecasting system 200 can be configured to perform “forward prediction,” e.g., by predicting the surface of the tissue region at a second time point that is after first time point, or “backward prediction,” e.g., by predicting the surface of the tissue region at a second time point that is before the first time point. The second time point can be separated from the first time point by any appropriate duration of time, e.g., one day, one week, one month, or one year.

In some implementations, the shape forecasting system 200 can be configured to predict the surface of the tissue region at a second time point that is separated from the first time point by a predefined duration of time, e.g., 1 year. In other implementations, the shape forecasting system 200 can be configured to receive an additional input that defines the second time point at which the surface of the tissue region is to be predicted recursively (as will be described in more detail below). In these implementations, the shape forecasting system 200 can receive data defining the second time point, e.g., through an application programming interface (API), or through a user interface made available by the shape forecasting system 200.

The shape forecasting system 200 can be configured to receive any of a variety of possible inputs in addition to the surface model of the tissue region at the first time point. For instance, the shape forecasting system 200 can receive an input that defines the second time point, i.e., at which the surface of the tissue region is to be predicted, as described above. As another example, the shape forecasting system can receive an input that defines one or more features of the subject, e.g., demographic features of the subject (e.g., age, gender, etc.), physiological measurements of the subject (e.g., blood pressure, cholesterol levels, weight, etc.), medical history of the subject (e.g., whether the subject smokes or drinks alcohol), etc.

The shape forecasting system 200 can be used in any of a variety of applications. A few example applications of the shape forecasting system 200 are described next.

In some implementations, after generating the predicted surface model of the tissue region at the second time point, the shape forecasting system 200 can process the predicted surface model at the second time point to generate a predicted surface model at a third time point (e.g., a recursive model to forecast surface changes further with respect to time). That is, instead of processing a surface model of the tissue region that is directly derived from a medical image of the tissue region, the shape forecasting system 200 can instead process a surface model of the tissue region that was previously generated by the shape forecasting system 200. Moreover, the operations of the shape forecasting system 200 can be iterated any appropriate number of times. For instance, the shape forecasting system 200 can “roll out” respective predictions for the surface of the tissue region at any appropriate number of time points, e.g., 2 time points, 5 time points, or 10 time points, by iteratively reprocessing predicted surface models. The shape forecasting system 200 can thus predict the surface of a tissue region at time points arbitrarily distant from the first time point.

In some implementations, the shape forecasting system 200 can process the predicted surface model of the tissue region at the second time point to classify the subject 102 into a class from a set of classes. For instance, the set of classes can include a “low-risk” class, e.g., indicating a low medical risk, and a “high-risk” class, e.g., indicating a high medical risk. Each class can be associated with a respective inclusion criterion, and the shape forecasting system 200 can designate the subject for inclusion in a class if the predicted surface of the tissue region satisfies the criterion for inclusion in the class. For instance, the shape forecasting system 200 can process the predicted surface of the tissue region to determine a predicted parameter (e.g., diameter or volume) of the tissue region at the second time point. The shape forecasting system can then classify the subject based at least in part on the predicted parameter of the tissue region at the second time point, e.g., based on whether the value of the predicted parameter satisfies a threshold.

A medical treatment (e.g., a drug, surgery, or other intervention) can be recommended or applied to the subject based at least in part on the classification of the subject (as described above). For instance, in response to the patient being classified into a “high-risk” class, e.g., based on a predicted future volume or diameter of a tumor of the subject, a drug (e.g., a chemotherapy drug) can be administered to the patient. As another example, in response to the patient being classified into a “high-risk” class, e.g., based on a predicted future volume or diameter of an aneurysm of the subject, a surgical procedure such as neurosurgical clipping or endovascular coiling can be performed on the subject. A recommended medical treatment can be presented to a clinician in any appropriate manner, including by displaying the recommendation, and optionally the classification, on an electronic display for viewing by the clinician. The clinician can then assess whether to administer the treatment based on the recommendation and other medically relevant factors.

In some implementations, the shape forecasting system 200 can process the predicted surface model of the tissue region at the second time point to predict a duration of time until a parameter of the tissue region, e.g., the volume or the diameter of the tissue region, satisfies a threshold. For instance, the shape forecasting system 200 can estimate the rate of the change of the parameter of the tissue region, e.g., as a ratio of: (i) the change in the value of the parameter between the first time point and the second time point, and (ii) the duration of time between the first time point and the second time point. The shape forecasting system can then determine the duration of time until the parameter of the tissue region is predicted to satisfy the threshold based on: (i) the value of the parameter of the tissue region at the first time point, and (ii) the predicted rate of change of the parameter of the tissue region.

In some implementations, the shape forecasting system can generate a visualization of the surface model of the tissue region at the second time point, and can cause the visualization to be presented on a user device, e.g., a computer, by way of a user interface, e.g., a graphical user interface.

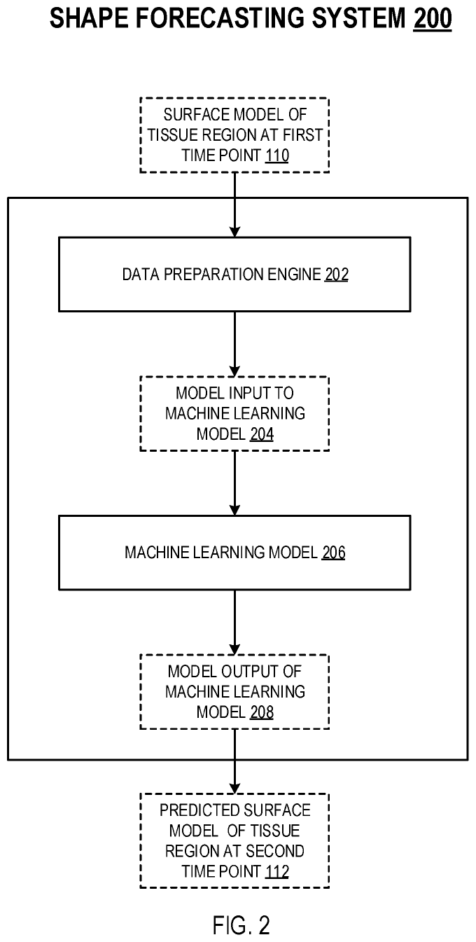

FIG. 2 shows an example shape forecasting system 200. The shape forecasting system 200 is an example of a system implemented as computer programs on one or more computers in one or more locations in which the systems, components, and techniques described below are implemented.

The shape forecasting system 200 is configured to process a surface model of a tissue region of a subject at a first time point 110 to generate a predicted surface model of the tissue region of the subject at a second time point 112. The surface model of the tissue region at the first time point can be, e.g., derived from one or more medical images of the subject, or generated as a previous output of the shape forecasting system 200, as described above with reference to FIG. 1. The shape forecasting system 200 can receive additional inputs, e.g., that define the second time point, or that define one or more features of the subject, e.g., demographic features, physiological measurements, medical history, etc.

The shape forecasting system 200 includes a data preparation engine 202 and a machine learning model 206, which are each described in more detail next.

The data preparation engine 202 is configured to process the surface model of the tissue region at the first time point 110 to generate a model input 204 to the machine learning model 206. The model input 204 to the machine learning model 206 can include a respective set of features corresponding to each surface point in a collection of surface points located on the surface of the tissue region at the first time point. To generate the model input 204 to the machine learning model 206, the data preparation engine 202 can identify a collection of surface points located on the surface of the tissue region, and generate a respective set of features corresponding to each surface point in the collection of surface points. The set of features corresponding to a surface point can include “point-specific” features, i.e., that are specific to the surface point, and optionally, “global” features, e.g., that characterize the tissue region or the subject as a whole.

The data preparation engine 202 can identify a collection of surface points located on the surface of the tissue region at the first time point in any of a variety of ways. In some cases, the surface model of the tissue region is represented as a point cloud, and the data preparation engine 202 can directly select some or all of the points in the point cloud as surface points. In some cases, the surface model of the tissue region is represented as a mesh, and the data preparation engine 202 can identify a collection of surface points on the surface of the tissue region by uniformly sampling points from across the mesh. Optionally, the data preparation engine 202 can be configured to identify a collection of surface points that includes a predetermined number of points, e.g., 20,000 points, to cause the model input 204 to the machine learning model 206 to have a fixed size (dimensionality).

The data preparation engine 202 can generate point-specific features for a surface point on the surface of the tissue region at the first time point in any of a variety of possible ways. A few examples of point-specific features corresponding to a surface point on the surface of the tissue region are described next.

In some implementations, the set of point-specific features for a surface point on the surface of the tissue region includes one or more features defining the coordinates of the surface point.

In some implementations, the set of point-specific features for a surface point on the surface of the tissue region includes one or more features defining a distance from the surface point to a centerline of a region. The centerline of a tissue region is generally located in an interior of the tissue region defined for the disease state. In some examples, the centerline can be determined as the center of fitting spheres inside the surface using a 2D/3D Voronoi approach. Smaller spheres are virtually packed in the surface and the center of spheroids are connected to define the centerline.

In some implementations, the set of point-specific features for a surface point on the surface of the tissue region includes one or more features defining a distance from the surface point to a center of mass of the region.

In some implementations, the set of point-specific features for a surface point on the surface of the tissue region includes one or more features characterizing a curvature of the surface in the vicinity of the surface point. For instance, to generate the set of point-specific features for a given surface point, the data preparation engine 202 can identify a predefined number of “neighboring” surface points (e.g., 5 points) having a minimum distance to the given surface point from among the collection of surface points (e.g., n nearest neighbors). The data preparation engine 202 can measure a respective distance of each neighboring surface point to the given surface point, to a centerline of the tissue region, to a center of mass of the tissue region, or a combination of these, and then define the measured distances as point-specific features of the given surface point. Intuitively, greater differences between the distances of neighboring surface points to the centerline or center of mass of the tissue region can be indicative of higher surface curvature at the given surface point. Likewise, where the surface model has a fixed number of points and the density of points in a localized area of the surface model reflects the amount of local curvature in the tissue region, shorter distances between the given point and its neighbors can indicate higher surface curvature at the given point and longer distances can indicate lower surface curvature at the given point.

In some implementations, the set of point-specific features for a surface point on the surface of the tissue region includes one or more features characterizing a stress (e.g., a biomechanical stress) at the surface point on the surface of the tissue region. The data preparation engine 202 can determine the stress at a surface point on the surface of the tissue region in any appropriate way. For instance, the data preparation engine 202 can determine the stress at a surface point on the surface of an aneurysm by performing a fluid dynamics simulation that models the flow of blood through the aneurysm. Techniques for predicting wall stress and other biomechanical indices of an aneurysm are further described in PCT/US2020/055511, filed Oct. 14, 2020 and published Apr. 22, 2021 as WO 2021/076575, the entire contents of which are incorporated by reference in their entirety into the disclosure of this specification.

The data preparation engine 202 can generate global features, e.g., that characterize the tissue region or the subject as a whole, in any of a variety of possible ways. A few examples of global features are described next.

In some implementations, the set of global features includes morphological features of the tissue region, e.g., that characterize the geometry of the tissue region as a whole. For instance, for an aneurysm, the data preparation engine 202 can generate morphological features including one or more of: a tortuosity of the aneurysm, features of the intraluminal thrombus (ILT) region such as a maximum, minimum, and/or average thickness of the ILT region, a volume of the aneurysm, a maximum, minimum, and/or average diameter of the outer wall and/or lumen of the aneurysm, an outer wall and/or luminal wall surface area. The data preparation engine 202 can generate morphological features of the tissue region by processing the surface model of the tissue region at the first time point.

In some implementations, the set of global features includes biomechanical features of the tissue region. For instance, for an aneurysm, the data preparation engine 202 can generate biomechanical features including, e.g., average wall stress or tension on the aneurysm, and peak wall stress or tension on the aneurysm. The data preparation engine 202 can generate biomechanical features of the aneurysm by subjecting the surface model to forces that simulate blood flow through the aneurysmal region of the aorta, and optionally additional forces that the aneurysm may experience in vitro. For example, the data preparation engine 202 can apply a pressure of 120 mmHg corresponding to typical systolic blood pressures as a force directed radially outward from the lumen. The surface model can be computationally defined with geometries, material properties, and material interactions that substantially mimic the actual geometries, material properties and interactions of the real tissue in the subject's aortic aneurysm. When the outward force from simulated blood flow is applied to the computational model, portions of the aneurysm may experience expansion or compression that deform the geometry of the aneurysm or otherwise result in stresses that could lead to rupture.

In some implementations, the set of global features includes features characterizing the subject, e.g., demographic features of the subject, physiological measurements of the subject, medical history of the subject, etc. The shape forecasting system 200 can obtain features characterizing the subject as an input, as described above.

In some implementations, the set of global features includes a feature defining the second time point, i.e., at which the surface of the tissue region is to be predicted. The second time point can be defined, e.g., by a feature specifying a duration of time between the first time point and the second time point, e.g., one day, one month, or one year.

The data preparation engine 202 can generate the model input 204 to the machine learning model 206 by combining the point-specific features with the global features. For instance, the data preparation engine 202 can concatenate the set of global features to the respective set of point-specific features for each surface point in the collection of surface points.

The machine learning model 206 is configured to process the model input 204, in accordance with values of a set of machine learning model parameters, to generate a model output 208 that defines the predicted surface model 112 of the tissue region at the second time point. More specifically, the model output 208 can include a respective output prediction for each surface point in the collection of surface points. The output predictions for the surface points in the collection of surface points collectively specify the predicted surface model of the tissue region at the second time point. A few examples of output predictions for surface points in the collection of surface points are described next.

In some implementations, the output prediction for each surface point defines a predicted position of the surface point at the second time point. Thus the model output 208 of the machine learning model 206 directly defines a point cloud of points that are predicted to be on the surface of the tissue region at the second time point.

In some implementations, the output prediction for each surface point defines a predicted displacement of the surface point between the first time point and the second time point. The displacement of a surface point between the first time point and the second time point can be represented, e.g., as a vector in an appropriate coordinate system. For each surface point, the shape forecasting system 200 can combine (e.g., sum): (i) the coordinates of the surface point at the first time point, and (ii) the predicted displacement of the surface point, to generate predicted coordinates of the surface point at the second time point. The predicted coordinates of the surface points at the second time point collectively define the predicted surface model of the tissue region at the second time point.

In some cases, the output prediction for each surface point defines a predicted displacement of the surface point over a fixed duration of time following the first time point. However, the second time point may be separated from the first time point by a multiple of the fixed duration of time. For instance, the fixed duration of time may be one month, while the second time point may be one year after the first time point, in which case the second time point is separated from the first time point by twelve multiples of the fixed duration of time. In this case, for each surface point, the shape forecasting system 200 can scale the predicted displacement of the surface point by the appropriate multiple prior to using the predicted displacement to generate the predicted coordinates of the surface point at the second time point, as described above.

Rather than generating a single predicted surface model for the second time point, the machine learning model 206 can optionally generate a family of multiple predicted surface models for the second time point. Generating a family of multiple predicted surface models can provide a means of interpreting underlying uncertainty in the predicted future state of the tissue region.

The machine learning model 206 can generate a family of multiple predicted surface models at the second time point in any of a variety of possible ways. For example, for each surface point, the machine learning model 206 can initially generate a probability distribution over a space of possible output predictions for the surface point. For instance, if the output prediction for a surface point is a displacement of the surface point between the first time point and the second time point, then the machine learning model 206 can generate a probability distribution over a space of possible displacements. The probability distribution can be, e.g., a Gaussian distribution over [symbol] 3. To generate a predicted surface model for the second time point, the machine learning model 206 can sample a respective output prediction for each surface point in the collection of surface points in accordance with the corresponding probability distribution over the space of possible output predictions. Each instance of sampling output predictions for the surface points in the collection of surface points results in a respective (different) predicted surface model for the second time point, i.e., due to the randomness of the sampling.

The machine learning model 206 can have any appropriate architecture that enables the machine learning model 206 to perform its described functions. For instance, the machine learning model can be implemented as a neural network, a random forest, a support vector machine, or as any other appropriate model. In implementations, where the machine learning model is implemented as a neural network, the neural network can include any appropriate types of neural network layers (e.g., fully connected layers, convolutional layers, attention layers, etc.) in any appropriate numbers (e.g., 5 layers, 10 layers, or 50 layers) and connected in any appropriate configuration (e.g., as a linear sequence of layers). In some implementations, the machine learning model is a transformer neural network. In other implementations, the machine-learning model is a recurrent neural network, e.g., a long short-term memory (LSTM) neural network. The LSTM or other type of recurrent neural network can be configured to process a sequence of inputs to generate a single prediction of the aneurysm's growth at a single future point in time or to generate a sequence of predictions of the aneurysm's growth over multiple future points in time. Each input in the sequence of inputs characterizes a point cloud or other surface model for the aneurysm or other tissue at a different point in time.

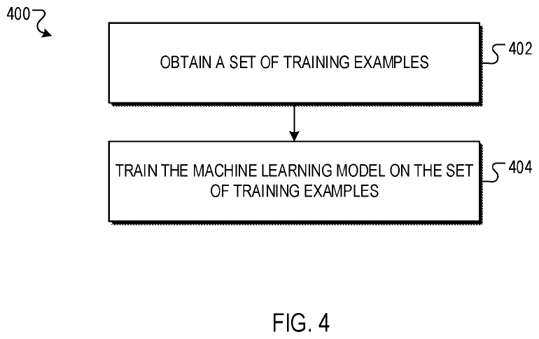

The shape forecasting system 200 can train the machine learning model 206, by a machine learning training technique, to determine trained values of a set of machine learning model parameters of the machine learning model. An example process for training the machine learning model is described in more detail with reference to FIG. 4.

FIG. 3 is a flow diagram of an example process 300 for predicting a surface model for a tissue region at a second time point based on a surface model for the tissue region at a first time point. For convenience, the process 300 will be described as being performed by a system of one or more computers located in one or more locations. For example, a shape forecasting system, e.g., the shape forecasting system 200 of FIG. 2, appropriately programmed in accordance with this specification, can perform the process 300.

The system obtains one or more medical images of the subject at a first time point (302). The medical images show a region of tissue in the subject, e.g., a tumor, an aneurysm, or a cyst. The medical images can be captured using any appropriate medical imaging modality, e.g., an MRI modality, a CT modality, an US modality, or an x-ray modality. The medical image can be a 2D medical image or a 3D medical image.

The system can segment or obtain a segmentation of the tissue region in the medical images (304). The segmentation can define, for each pixel in the medical image, whether the pixel is included in the tissue region. The segmentation can be manually generated by a user, e.g., using a manual or semi-automated segmentation software. The segmentation can also be generated in a fully automated manner, e.g., using a segmentation machine learning model that has been trained to perform tissue region segmentation.

The system generates data defining the surface of the tissue region at the first time point (306). The surface model of the tissue region can be represented, e.g., as a collection of points, or as a mesh. Example techniques for obtaining medical images, segmenting medical images, and generating surface models of tissue regions are described in more detail with reference to International Patent Application Serial No. PCT/US2022/051718, the entire contents of which are incorporated herein by reference.

The system generates a model input to the machine learning model based on the data defining the surface of the tissue region at the first time point (308). For example, to generate the model input, the system can identify a collection of surface points on the surface of the tissue region at the first time point. The system can then process the surface model for the first time point to generate a set of features for each surface point in the collection of surface points. In some implementations, for each surface point in the collection of surface points, the set of features for the surface point defines coordinates of the surface point at the first time point. In some implementations, for each surface point in the collection of surface points, the set of features for the surface point characterize a curvature of the surface of the tissue region at the surface point. In some implementations, for each surface point in the collection of surface points, the set of features for the surface point characterize a stress on the surface of the tissue region at the surface point.

Optionally, the model input to the machine learning model can include one or more morphological features that characterize a geometry of the tissue region, e.g., a tortuosity of the tissue region, a volume of the tissue region, or a thickness of a wall of the tissue region.

Optionally, the model input to the machine learning model can include one or more biomechanical features of the tissue region, e.g., that characterize an average wall stress or tension of the tissue region, or a peak wall stress or tension of the tissue region.

The system processes the model input using a machine learning model, in accordance with values of a set of machine learning model parameters, to generate a model output that specifies the predicted surface of the tissue region in the subject at a second time point (310). In some implementations, the model output of the machine learning model includes, for each surface point in the collection of surface points, a predicted displacement of the surface point between the first time point and the second time point. In some implementations, the model output of the machine learning model includes, for each surface point in the collection of surface points, predicted coordinates of the surface point at the second time point.

The system provides data defining the predicted surface of the tissue region in the subject at the second time point as an output (312). In some implementations, the system generates a second model input to the machine learning model based on the predicted surface model of the tissue region at the second time point. The system then processes the second model input using the machine learning model to generate a model output that specifies a predicted surface model of the tissue region in the subject at a third time point.

FIG. 4 is a flow diagram of an example process 400 for training a machine learning model that processes a model input based a surface model of a tissue region at a first time point to generate a model output that specifies a predicted surface model of the tissue region at a second time point. For convenience, the process 400 will be described as being performed by a system of one or more computers located in one or more locations. For example, a shape forecasting system, e.g., the shape forecasting system 200 of FIG. 2, appropriately programmed in accordance with this specification, can perform the process 400.

Before the model can be trained to generate predictions, data must be pre-processed and provided in a format that the neural network can accept for processing. The original 3D scans of a patient's AAA (or other tissue region) are converted to point cloud representations, with a respective point cloud representation of the AAA generated at multiple points in time corresponding during its growth (e.g., point clouds representing the AAA from scans taken every 6 months).

As more training data becomes available, the model can be trained to generate increasingly accurate predictions of the aneurysm's growth over time. The number of scans of patients' AAAs (or other tissues/tissue regions) may be limited, however. In some implementations, the volume of training data can be increased by generating point clouds for a patient's AAA (or other tissues/tissue regions) at additional points in time between the times at which scans were obtained using interpolation techniques to estimate the displacement and coordinates of each point during at intermediate times between scans. The surface models at the intermediate points in time can then processed to generate model inputs and/or used as target outputs for training the model.

In some examples, every point cloud has a fixed number of points (e.g., 24,661 points). Once sufficient data has been generated through the creation of artificial point clouds, the data can be provided to the training algorithm. For each point cloud, the coordinates of each point are written into a respective file or data structure that characterizes the point cloud for the patient's AAA (or other tissue/tissue region) at a particular point in time during growth of the AAA (or other tissue/tissue region). The coordinates can be expressed in any suitable manner, e.g., as cartesian or polar coordinates. Along with coordinates for each point, the system can further generate one or more vectors containing values that indicate a direction and/or magnitude of each point's displacement over time and that identifies each point's respective n nearest neighbors (e.g., 1, 2, 5, 10, 15, or 20 nearest neighbors) in the point cloud. The coordinates of each point's n nearest neighbors can also be recorded for each point. The coordinates, displacement, and nearest neighbor data collectively indicate how each section of the aneurysm morphs over time and provides a baseline from which a forecasted shape can be predicted. In general, any of the “point-specific” or “global” features of the surface model described in this specification can be included in the model inputs used in the training data.

After training data is generated, the model is provided with the necessary inputs from each training example to generate predictions. The model can be provided with all or a subset of available features (the coordinates, displacement vector, and nearest neighbors of each point). Applying a regression algorithm, the model can analyze corresponding points from each point cloud at each interval in time. Considering the vector that each point travels over time during growth of the aneurysm, the model generates a prediction of where that point is likely to be located at a future point in time, e.g., x months into the future. From there, the model can output predicted displacements for each point which would then be used to generate a new predicted point cloud and reconstruct a 3D depiction of the aneurysm to determine how severe the risk of rupture is at that future point in time.

Referring again to FIG. 4, the system obtains a set of training examples (402). Each training example corresponds to a training subject and includes: (i) a model input to the machine learning model, and (ii) a target output of the machine learning model. The model input to the machine learning model is based on surface model of a tissue region in the training subject at a first time point. The model input can include a respective set of features (including point-specific features, and optionally, global features) for each surface point in a collection of surface points of the tissue region in the training subject at the first time point. The target output can define the model output that should be generated by the machine learning model by processing the model input of the training example. For instance, the model output can include a respective output target for each surface point, e.g., that defines a displacement of the surface point between the first time point and a second time point.

The system can generate a training example corresponding to a training subject by processing: (i) a medical image of the tissue region in the training subject at the first time point, and (ii) a medical image of the tissue region in the training subject at the second time point. More specifically, the system can process the medical image of the tissue region at the first time point to segment the tissue region and then generate a first surface model of the tissue region at the first time point, e.g., as a point cloud. Similarly, the system can process the medical image of the tissue region at the second time point to segment the tissue region and then generate a second surface model of the tissue region at the second time point, e.g., as a point cloud. The system can then map each surface point on the first surface model to a corresponding surface point on the second surface, e.g., using a point-to-point minimization algorithm. More specifically, the point-to-point minimization algorithm can determine an assignment of surface points from the first surface model to surface points on the second surface model that (approximately or exactly) minimizes an objective function. The objective function can measure distances between surface points on the first surface model and their assigned surface points on the second surface model.

The system can generate the training examples, e.g., by processing medical record data for a set of training patients. The system can generate any appropriate number of training examples, e.g., 100 training examples, 1,000 training examples, or 100,000 training examples. In some implementations, the system can create more training examples than the number of sets of medical images available or the number of patients' tissue regions that have been imaged. The additional training examples can be created, for example, by interpolating or extrapolating additional surface models from the surface models that were generated directly from medical images. For example, using a linear interpolation technique, an interpolated surface model at a third time point between a first time point and a second time point can be created by taking a weighted or non-weighted average of the coordinates of each registered point on the surface models to obtain an interpolated coordinate for the point at the third time point. The collection of interpolated points forms the interpolated surface model. A new training example can then be created by deriving features and creating a model input from the interpolated surface model in substantially the same manner as the model input is generated from a non-interpolated surface model. The interpolated surface model can additionally or alternatively be used as a target output of the machine-learning model at the third time point. The model input or target output for the interpolated surface model can be paired with another target output or model input of either an interpolated or non-interpolated surface model, respectively, to form a training example.

In some implementations, the interval between the time point of the model input and the time point of the target output in a training example is fixed across training examples (e.g., 3 months, 6 months, 9 months, 12 months, 18 months, or 24 months) so that the machine-learning model is trained to predict the geometry of the tissue region a fixed interval of time in the future (e.g., 6 months, 9 months, 12 months, 18 months, or 24 months after the time point represented by the model input). In other implementations, the time intervals between the surface models represented in the model input and the target output in different training examples can vary and the model input can further include a time value that indicates the length of the particular time interval between the model input and the target output in that training example. The time value is then processed by the machine-learning model so that the model learns to forecast the shape of the tissue region at different future points in time indicated by the time value in the model input.

The system trains the machine learning model on the set of training examples (404). For each training example, the system trains the machine learning model to process the model input of the training example to generate a model output that matches the target output of the training example. More specifically, the system trains the machine learning model to optimize an objective function that measures, for each training example, an error between: (i) the target output of the training example, and (ii) the model output generated by the machine learning model by processing the model input of the training example. The objective function can measure the error between a target output and a model output, e.g., using any appropriate numerical norm, e.g., an L₁ norm or an L₂ norm. The system can train the machine learning model using any appropriate machine learning training technique, e.g., stochastic gradient descent.

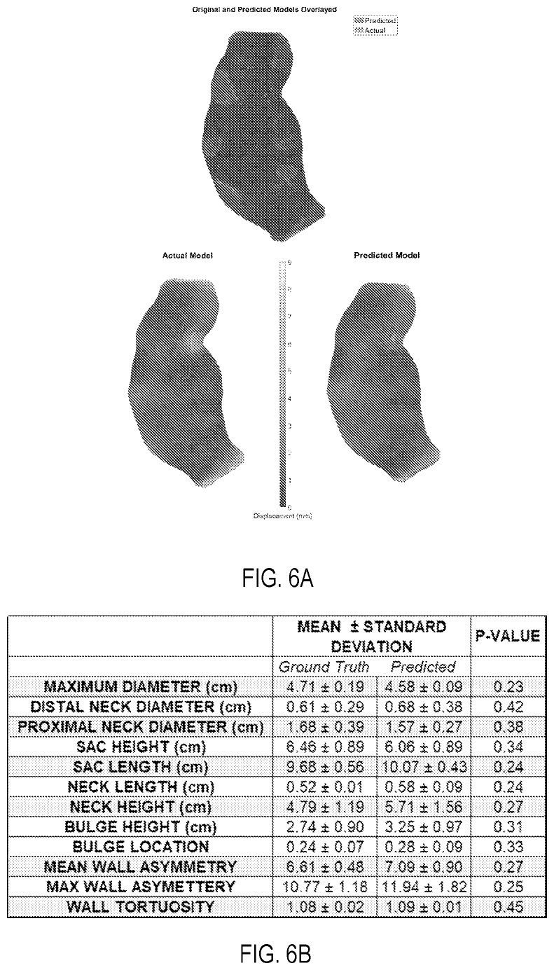

FIG. 6A depicts a comparison of a predicted surface model and an actual surface model (e.g., a target output) of an AAA in a study of an actual machine-learning model implemented according to the techniques disclosed in this specification. FIG. 6B is a table of values that indicate the accuracy of the predicted surface model with respect to the actual surface model (ground truth). The p-values for 1-tail heteroscedastic t-tests are shown for each metric to assess any significance in differences between the models. In this study, (1) a MATLAB script was used to generate point clouds (nodes based in Cartesian coordinates) from patient scans, (2) linear interpolation was used to create nodal clouds at intermediate timepoints, (3) a machine-learning programmed in TENSORFLOW, KERAS, or PYTORCH, used half of the available data (80/20 split) to train and test the model, (4) an LSTM architecture was used for temporal predictions, (5) the machine-learning model was used to predict the final half of the training data, (6) the predicted nodal displacements were validated based on the linear interpolated surface at the final timepoint, and (7) a post-processing MATLAB or PYTHON script can be used to visualize the 3D surface predictions for growth and assess accuracy of predictions. Additionally, outliers of the 3D point cloud can be removed prior to meshing to clean up the visualization of the data using well-established algorithms that remove outliers from scattered datasets.

A local workstation with a computer processor and/or graphics processor unit optimized for machine learning or deep learning approaches can be used to calculate the surface changes, train relevant models, and feed input data into the trained models to extract predictions of surface changes with time. A cloud-based computing platform may also be used that combines CPU and GPU processing power to perform the aforementioned computations.

This specification uses the term “configured” in connection with systems and computer program components. For a system of one or more computers to be configured to perform particular operations or actions means that the system has installed on it software, firmware, hardware, or a combination of them that in operation cause the system to perform the operations or actions. For one or more computer programs to be configured to perform particular operations or actions means that the one or more programs include instructions that, when executed by data processing apparatus, cause the apparatus to perform the operations or actions.

Embodiments of the subject matter and the functional operations described in this specification can be implemented in digital electronic circuitry, in tangibly-embodied computer software or firmware, in computer hardware, including the structures disclosed in this specification and their structural equivalents, or in combinations of one or more of them. Embodiments of the subject matter described in this specification can be implemented as one or more computer programs, i.e., one or more modules of computer program instructions encoded on a tangible non-transitory storage medium for execution by, or to control the operation of, data processing apparatus. The computer storage medium can be a machine-readable storage device, a machine-readable storage substrate, a random or serial access memory device, or a combination of one or more of them. Alternatively, or in addition, the program instructions can be encoded on an artificially-generated propagated signal, e.g., a machine-generated electrical, optical, or electromagnetic signal, that is generated to encode information for transmission to suitable receiver apparatus for execution by a data processing apparatus.

The term “data processing apparatus” refers to data processing hardware and encompasses all kinds of apparatus, devices, and machines for processing data, including by way of example a programmable processor, a computer, or multiple processors or computers. The apparatus can also be, or further include, special purpose logic circuitry, e.g., an FPGA (field programmable gate array) or an ASIC (application-specific integrated circuit). The apparatus can optionally include, in addition to hardware, code that creates an execution environment for computer programs, e.g., code that constitutes processor firmware, a protocol stack, a database management system, an operating system, or a combination of one or more of them.

A computer program, which may also be referred to or described as a program, software, a software application, an app, a module, a software module, a script, or code, can be written in any form of programming language, including compiled or interpreted languages, or declarative or procedural languages; and it can be deployed in any form, including as a stand-alone program or as a module, component, subroutine, or other unit suitable for use in a computing environment. A program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, sub-programs, or portions of code. A computer program can be deployed to be executed on one computer or on multiple computers that are located at one site or distributed across multiple sites and interconnected by a data communication network.

In this specification the term “engine” is used broadly to refer to a software-based system, subsystem, or process that is programmed to perform one or more specific functions. Generally, an engine will be implemented as one or more software modules or components, installed on one or more computers in one or more locations. In some cases, one or more computers will be dedicated to a particular engine; in other cases, multiple engines can be installed and running on the same computer or computers.

The processes and logic flows described in this specification can be performed by one or more programmable computers executing one or more computer programs to perform functions by operating on input data and generating output. The processes and logic flows can also be performed by special purpose logic circuitry, e.g., an FPGA or an ASIC, or by a combination of special purpose logic circuitry and one or more programmed computers.

Computers suitable for the execution of a computer program can be based on general or special purpose microprocessors or both, or any other kind of central processing unit. Generally, a central processing unit will receive instructions and data from a read-only memory or a random access memory or both. The essential elements of a computer are a central processing unit for performing or executing instructions and one or more memory devices for storing instructions and data. The central processing unit and the memory can be supplemented by, or incorporated in, special purpose logic circuitry. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few.

Computer-readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's device in response to requests received from the web browser. Also, a computer can interact with a user by sending text messages or other forms of message to a personal device, e.g., a smartphone that is running a messaging application, and receiving responsive messages from the user in return.

Data processing apparatus for implementing machine learning models can also include, for example, special-purpose hardware accelerator units for processing common and compute-intensive parts of machine learning training or production, i.e., inference, workloads.

Machine learning models can be implemented and deployed using a machine learning framework, e.g., a TensorFlow framework, or a Jax framework.

Embodiments of the subject matter described in this specification can be implemented in a computing system that includes a back-end component, e.g., as a data server, or that includes a middleware component, e.g., an application server, or that includes a front-end component, e.g., a client computer having a graphical user interface, a web browser, or an app through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (LAN) and a wide area network (WAN), e.g., the Internet.

The computing system can include clients and servers. A client and server are generally remote from each other and typically interact through a communication network. The relationship of client and server arises by virtue of computer programs running on the respective computers and having a client-server relationship to each other. In some embodiments, a server transmits data, e.g., an HTML page, to a user device, e.g., for purposes of displaying data to and receiving user input from a user interacting with the device, which acts as a client. Data generated at the user device, e.g., a result of the user interaction, can be received at the server from the device.

While this specification contains many specific implementation details, these should not be construed as limitations on the scope of any invention or on the scope of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially be claimed as such, one or more features from a claimed combination can in some cases be excised from the combination, and the claimed combination may be directed to a subcombination or variation of a subcombination.

Similarly, while operations are depicted in the drawings and recited in the claims in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

What is claimed is:

1. A method performed by one or more computers, the method comprising:
obtaining a surface model that defines a surface of a tissue region in a subject at a first time point;
generating a predicted surface model that defines a predicted surface of the tissue region in the subject at a second time point using a machine learning model, comprising:
generating a model input to the machine learning model based on the surface model of the tissue region at the first time point; and
processing the model input using the machine learning model, in accordance with values of a set of machine learning model parameters, to generate a model output that specifies the predicted surface model of the tissue region in the subject at the second time point; and
providing the predicted surface model of the tissue region in the subject at the second time point as an output.

2. The method of claim 1, wherein generating the model input to the machine learning model based on the surface model of the tissue region at the first time point comprises:
identifying a collection of surface points on the surface of the tissue region at the first time point; and
processing the surface model for the first time point to generate a set of features for each surface point in the collection of surface points.

3. The method of claim 2, wherein for each surface point in the collection of surface points, the set of features for the surface point include spatial coordinates f the surface point at the first time point.

4. The method of claim 2, wherein for each surface point in the collection of surface points, the set of features for the surface point characterize a curvature of the surface of the tissue region at the surface point.

5. The method of claim 2, wherein for each surface point in the collection of surface points, the set of features for the surface point characterize a stress on the surface of the tissue region at the surface point.

6. The method of claim 1, wherein the model input to the machine learning model includes one or more morphological features that characterize a geometry of the tissue region.

7. The method of claim 6, wherein the morphological features comprise one or more of: a feature characterizing a tortuosity of the tissue region, a feature characterizing a volume of the tissue region, or a feature characterizing a thickness of a wall of the tissue region.

8. The method of claim 1, wherein the model input to the machine learning model includes one or more biomechanical features of the tissue region.

9. The method of claim 8, wherein the biomechanical features of the tissue region comprise one or more of: a feature characterizing an average wall stress or tension of the tissue region, or a feature characterizing a peak wall stress or tension of the tissue region.

10. The method of claim 2, wherein the model output of the machine learning model comprises, for each surface point in the collection of surface points, a predicted displacement of the surface point between the first time point and the second time point.

11. The method of claim 2, wherein the model output of the machine learning model comprises, for each surface point in the collection of surface points, predicted coordinates of the surface point at the second time point.

12. The method of claim 1, wherein the machine learning model comprises a neural network model.

13. The method of claim 1, wherein the machine learning model has been trained by operations comprising:
obtaining a set of training examples, wherein each training example comprises: (i) a training input to the machine learning model based on a surface model of a tissue region in a training subject at a first time point, and (ii) a target output that specifies a surface model of the tissue region in the training subject at a second time point; and
training the machine learning model on the set of training examples, by a machine learning training technique, to determine trained values of the set of machine learning model parameters.

14. The method of claim 13, wherein training the machine learning model on the set of training examples comprises, for each training example:
training the machine learning model to process to the training input of the training example to generate a model output that matches the target output of the training example.

15. The method of claim 1, wherein obtaining the surface model that defines the surface of the tissue region in the subject at the first time point comprises:
obtaining a medical image of the subject at the first time point, wherein the medical image shows the tissue region;
processing the medical image to generate a segmentation of the tissue region in the medical image; and
processing the segmentation of the tissue region in the medical image to generate the surface model of the tissue region at the first time point.

16. The method claim 1, wherein providing the predicted surface model of the tissue region in the subject at the second time point as an output comprises:
generating a second model input to the machine learning model based on the predicted surface model of the tissue region at the second time point; and
processing the second model input using the machine learning model to generate a model output that specifies a predicted surface model of the tissue region in the subject at a third time point.

17. The method of claim 1, wherein providing the predicted surface model of the tissue region in the subject at the second time point as an output comprises:
generating a visualization of the predicted surface model of the tissue region in the subject at the second time point; and
providing the visualization for display on a user device.

18. (canceled)

19. (canceled)

20. (canceled)

21. The method of claim 1, wherein the tissue region comprises a tumor.

22. (canceled)

23. A system comprising:
one or more computers; and
one or more storage devices communicatively coupled to the one or more computers, wherein the one or more storage devices store instructions that, when executed by the one or more computers, cause the one or more computers to perform operations of a method, wherein the method comprises:
obtaining a surface model that defines a surface of a tissue region in a subject at a first time point;
generating a predicted surface model that defines a predicted surface of the tissue region in the subject at a second time point using a machine learning model, comprising:
generating a model input to the machine learning model based on the surface model of the tissue region at the first time point; and
processing the model input using the machine learning model, in accordance with values of a set of machine learning model parameters, to generate a model output that specifies the predicted surface model of the tissue region in the subject at the second time point; and
providing the predicted surface model of the tissue region in the subject at the second time point as an output.

24. One or more non-transitory computer storage media storing instructions that when executed by one or more computers cause the one or more computers to perform operations of a method, wherein the method comprises:
obtaining a surface model that defines a surface of a tissue region in a subject at a first time point;
generating a predicted surface model that defines a predicted surface of the tissue region in the subject at a second time point using a machine learning model, comprising:
generating a model input to the machine learning model based on the surface model of the tissue region at the first time point; and
processing the model input using the machine learning model, in accordance with values of a set of machine learning model parameters, to generate a model output that specifies the predicted surface model of the tissue region in the subject at the second time point; and
providing the predicted surface model of the tissue region in the subject at the second time point as an output.
